(19) United States
(12) Patent Application Publication
(10) Pub. No.: US 2002/0081574 A1
(43) Pub. Date: Jun. 27, 2002

(54) Methods for identifying inhibitors of helicase C virus

(76) Inventors: Marc S. Collett, Collegeville, PA (US); Daniel C. Pevear, Harleysville, PA (US); James M. Groarke, Phoenixville, PA (US); Dorothy C. Young, Collegeville, PA (US)

Correspondence Address: DANN DORFMAN HERRELL & SKILLMAN, SUITE 720, 1601 MARKET STREET, PHILADELPHIA, PA, 19103-2307, US

(21) Appl. No.: 10/010,860

(22) Filed: Dec. 7, 2001

Related U.S. Application Data
(63) Continuation of application No. 08/678,771, filed on Jul. 11, 1996, now abandoned.
(60) Provisional application No. 60/010,474, filed on Jan. 23, 1996.

(51) Int. Cl. C12Q 1/70; C12N 9/22; C12N 15/86; C12P 21/02

(52) U.S. Cl. 435/5; 435/69.1; 435/199; 435/456

(57) ABSTRACT

Assay methods are disclosed having utility in the identification of therapeutic agents effective against human hepatitis C virus and related viruses. NS3 protein derived from these viruses, representing a native and authentic version of the complete protein sequence, is used in the assay methods of the invention, which enables identification and development of anti-viral agents exhibiting inhibition of the protein's NTPase and RNA helicase activities.

This application is a continuation of U.S. patent application Ser. No. 08/678,771 filed Jul. 11, 1996, which in turn claims priority from U.S. Provisional Application No. 60/010,474 filed Jan. 23, 1996, the entire disclosure of each being incorporated by reference herein.

### FIELD OF THE INVENTION

The present invention relates to the fields of molecular biology and biochemistry. More specifically, the invention provides materials and methodology for the identification and development of agents capable of inhibiting the essential nucleoside triphosphatase (NTPase) and RNA helicase activities of certain RNA viruses, particularly human hepatitis C and related viruses.

### BACKGROUND OF THE INVENTION

Several publications are referenced in this application by numerals in parenthesis in order to more fully describe the state of the art to which this invention pertains, as well as to aid in describing the invention itself. Full citations for these references are found at the end of the specification. Each of these publications is incorporated herein by reference.

Non-A non-B hepatitis (NANBH) is a major cause of morbidity and mortality throughout the world. The principal etiologic agent of NANBH is hepatitis C virus (HCV)(1). HCV has an estimated worldwide prevalence of 0.5-1.5% and can establish a life-long asymptomatic carrier state. About 80% of infected persons will develop chronic hepatitis; 20% of these will go on to develop cirrhosis of the liver. Chronic HCV infection, over a period of 20 to 30 years, can lead to development of hepatocellular carcinoma. The pathogenic mechanisms that allow persistence and the high rate of chronic liver disease are not yet understood. Nor is it known how HCV interacts with, and evades, the host immune system. Additionally, the roles of cellular and humoral immune responses in protection against HCV infection and disease, or in enhancement or exacerbation of infection and disease, have yet to be established.

HCV is an enveloped positive strand RNA virus in the Flaviviridae family (2). In addition to HCV, this virus family includes the flavivirus genus, which consists of a number of viruses pathogenic to humans such as the dengue fever viruses and various encephalitis viruses. Also included in the Flaviviridae family is the pestivirus genus, representatives of which are the animal pathogens bovine viral diarrhea virus, classical swine fever virus, and border disease virus. These viruses are responsible for large economic losses in the livestock industry. Finally, the newly discovered viruses, hepatitis G virus (HGV) and hepatitis GB virus, are provisionally considered to be members of the same family (2-4).

Viruses within the Flaviviridae family share many characteristics (2). For HCV, the single strand RNA genome is approximately 9.4 kilobases (kb) in length and has a single large open reading frame (ORF) encoding about 3000 amino acids. Coding assignments of the mature proteins within the ORF are as follows:

NH₂-[C-E1-E2-p7-NS2B-NS3-NS4A-NS4B-NS5A-NS5B]-COOH

The C (nucleocapsid or core protein), E1, and E2 (two envelope glycoproteins) represent the putative viral structural proteins. It is not yet clear if the p7 protein is structural or nonstructural. These structural polypeptides are followed by the viral nonstructural (NS) proteins. The NS proteins of viruses of the Flaviviridae are thought to be essential for viral gene expression and RNA replication. Enzymatic activities have been ascribed to several of these NS proteins. In particular, the NS3 protein, in association with the NS2B and NS4A proteins, possesses two distinct proteinase activities (5-10). NS3 is also a nucleoside triphosphatase (NTPase) (11-14) and RNA helicase (15-17, and as described herein). Although not yet experimentally established, it is likely the NS5A and NS5B proteins make up a component of the viral RNA replicase.

While many viruses may be propagated in cells in culture relatively effectively, HCV can be propagated in vitro only with difficulty. In this regard, see published European Patent Application No. 0414475. However, numerous HCV isolates have been molecularly cloned and sequenced. Comparisons among HCV nucleotide sequences have demonstrated that the viral genome exhibits considerable genetic heterogeneity. This heterogeneity has been categorized in two types: “quasispecies”, referring to sequence variation in the virus population within an infected individual, and “genotypes”, indicating sequence heterogeneity among different HCV isolates. Quasispecies sequence variation is based on multiple mutations found in a hypervariable region of the E2 protein (18-20). Genotypic sequence variation is thought to be the consequence of the accumulation of mutations, distributed throughout the viral genome, during the independent evolution of virus isolates. Comparison among the sequences of HCV isolates has resulted in the classification of HCV into 9 distinct genotypes and at least 30 subtypes (21). The sequence diversity between members of the same genotype is generally less than 6%, while the differences in nucleotide sequences between isolates of different genotypes ranges from 11 to 33% (21-24).

HCV may be associated with either mild or severe disease and thus it is believed that HCV genetic variation plays a role in disease progression. For example, genotype 2a is associated with mild histologic forms of chronic hepatitis, while genotype 1b is more frequently found in chronic liver disease and is more frequently observed in advanced liver diseases, such as cirrhosis and hepatocellular carcinoma (21, 25). Also, the diversity of HCV quasispecies becomes more complex with the stage of liver disease, further suggesting an association with disease progression (26, 27).

For treatment of hepatitis due to HCV, interferon alpha (IFN-α) is currently the only approved drug in the U.S. IFN-β is approved in Japan. IFN treatment is associated with improved serum liver enzyme response in 20-40% of patients. The remainder are nonresponsive to IFN treatment. For responders, a sustained improvement of aminotransferase levels is seen in only 10-20% of patients; the majority of patients relapse upon cessation of IFN-α treatment. The outcome of IFN therapy may be related to the HCV genotype with which the patient is infected (21, 22). Generally, infection with genotype 1b is associated with a poor response to IFN therapy, while high sustained response rates are seen in patients infected with genotypes 2a and 2b. Nonresponders to IFN were found to have greater quasispecies diversification than responders, implying that quasispecies evolution may contribute to the high rate of resistance of HCV to IFN therapy (21, 27-29). In those responsive to IFN treatment, it is not clear if the drug is acting directly as an antiviral or via some immunomodulatory mechanism. Thus, while IFN-α represents the first treatment of chronic hepatitis C, its effectiveness is variable, its cure rate is low, and associated adverse effects are considerable.

Vaccines under development for HCV generally consist of recombinant versions of the putative viral structural proteins (C, E1, E2), or the genes encoding such proteins. It is believed that virus neutralizing antibodies do exist, can be elicited, and may be able to inhibit or prevent HCV infection (30, 31). Initial challenge experiments in chimpanzees suggest that some protection can be afforded by vaccination (32). However, different viruses with immunologically distinct envelope proteins are not neutralized by pre-existing antibodies (31). Quasispecies diversification may represent a mechanism by which the virus escapes immune surveillance and establishes a persistent infection (33).

The protease necessary for polyprotein processing in Hepatitis C has been identified, cloned and used in assays for the design of therapeutic compounds effective against Hepatitis C. See WO 91/15575. Insofar as it is known, however, anti-viral assays based on the NTPase/helicase activities of HCV have not previously been developed.

HCV infection causes a debilitating illness, and while some forms of therapy are available, to date, an effective cure or treatment has not been found. There exists a need for the identification and development of agents capable of inhibiting essential enzymatic activities associated with this and other RNA viruses. The present invention provides materials and methodology designed to facilitate the identification and biochemical characterization of novel anti-viral compounds to beneficially augment those already available to treat illness associated with human hepatitis C virus and other related viruses.

### SUMMARY OF THE INVENTION

According to one aspect of the invention there is provided an RNA virus-encoded enzyme with NTPase and RNA helicase activity suitable for use as a target for antiviral drug assays, specifically, the NS3 protein of viruses within the Flaviviridae family. Methods and processes for the production and preparation of a full length, authentic sequence, of said enzymatically active protein are also within the scope of this invention.

According to another aspect, the present invention provides methods and processes for assaying putative anti-viral agents for their ability to inhibit the NTPase and/or the RNA helicase activities of RNA viruses, in particular, of human hepatitis C and other related viruses. In a particularly preferred embodiment, the assay method of the invention is efficiently utilized for screening of multiple putative anti-viral compounds simultaneously.

Enzymatically active NTPase/RNA helicase protein in its native form is prepared by molecularly cloning into standard plasmid vectors, the gene encoding the complete and authentic protein. The gene encoding the enzyme is then inserted into a suitable eukaryotic expression vector or expression system using standard recombinant DNA techniques to allow for the expression of the authentic protein in its native conformation. Once expressed, the protein is obtained from cells by employing protein extraction procedures that maintain the native conformation of the desired enzyme. The enzyme is then purified from such extract by procedures that preserve the native conformation of the enzyme. Once purified, the enzymatic activities associated with the protein are optimized with respect to enzyme reaction conditions and are quantitatively measured by suitable methods e.g., in the identification of anti-viral compounds.

With the appropriately produced and prepared enzyme and optimized enzyme reactions, a suitable biochemical assay has been developed that allows for the sensitive and quantitative measurement of enzyme activity. Adaptation of this assay to a format suitable for high capacity screening allows for the evaluation of large numbers of agents to determine their potential for inhibiting the enzyme activity. The combined use of such enzyme reagents, reaction conditions, and assays enables the efficient identification of anti-viral agents and compounds, which when appropriately formulated, are useful for the prevention and/or treatment of infections and diseases mediated by certain RNA viruses.

### BRIEF DESCRIPTION OF THE DRAWINGS

FIG. 1 is a schematic depiction of the 3011 amino acid open reading frame of the HCV genome indicating the protein coding regions for all currently recognized viral gene products, and NS3 protein coding regions expressed in various recombinant expression systems.

FIG. 2 presents the results of the immunoaffinity purification of the HCV NS3 protein derived from bacNS3-infected and bacNS3-5B-infected insect cells. A Coomassie-blue stained SDS-PAGE gel showing the electrophoretic mobility of material eluted with KSCN from a column to which human IgG containing HCV anti-NS3 antibodies has been covalently bound. Shown are independent eluates of starting material lysates derived from wild-type Autographa californica nuclear polyhedrosis virus (AcNPV)-infected Sf9 cells (lane 1); recombinant baculovirus bacNS3-infected (lane 2) and bacNS3-5B-infected Sf9 cells (lane 3). Molecular mass standards in kilodaltons are indicated at the left and the position of NS3 protein at the right.

FIG. 3 shows the ATPase and RNA helicase activities associated with the immunoaffinity purified materials depicted in FIG. 2. Panel A is an autoradiogram of a polyethyleneimine cellulose thin layer chromatography sheet after development in 0.375 M potassium phosphate, pH 3.5. Samples applied to the sheet were aliquots of ATPase reactions that were composed of α³²P-ATP (0.5 μCi) 200 μM unlabeled ATP, 50 mM PIPES, pH 6.5, 1 mM dithiothreitol, 100 μg/ml bovine serum albumin, 3 mM MnCl₂, 1 U/ml RNAsin and either no protein (lane 1), the AcNPV eluate (lanes 2 & 3), the bacNS3 eluate at approximately 60 ng/mL NS3 protein (lanes 4 & 5), or the bacNS3-5B eluate at approximately 60 ng/ mL NS3 protein (lanes 6 & 7). The reaction mixtures of lanes 1,3,5,7 were further supplemented with the homopolymer nucleic acid poly (U) at 100 μg/ml. Panel B is a graphical representation of the data depicting the time course of ATPase activity in ATPase reactions described in panel A by the AcNPV eluate (∘, ●), the bacNS3 eluate (Δ, ▴) and the bacNS3-5B eluate (□, ▪). Solid symbols in the graph indicate data derived from reactions to which poly (U) was added, open symbols refer to experiments in which poly(U) was omitted. Panel C is an autoradiographic image of polyacrylamide gel in which aliquots of RNA helicase reaction mixtures were subjected to electrophoresis. Helicase reactions consisted of 50 mM PIPES, pH 6.5, 1 mM dithiothreitol, 100 μg/ml bovine serum albumin, 3 mM MnCl₂, 1 mM ATP, 1 U/ml RNAsin, approximately 30 ng/ mL standard RNA substrate and either no protein (lanes 1 & 2), the AcNPV eluate (lane 3), the bacNS3 eluate at approximately 60 ng/mL NS3 protein (lane 4), or the bacNS3-5B eluate at approximately 60 ng/mL NS3 protein (lane 5). Lane 1 represents a sample of the RNA helicase substrate that was boiled prior to electrophoresis to denature the duplex structure and reveal the position in the gel of the release strand product. Panel D shows a plot of the data depicting the time course of RNA helicase activity in standard RNA helicase reactions described in Panel B by AcNPV eluate (∘), the bacNS3 eluate (▴), and the bacNS3-5B eluate (▪).

FIG. 4 depicts an autoradiographic image of a polyacrylamide gel showing the RNA helicase activity associated with an amino-terminal truncated YFV NS3 protein. The E. coli-produced protein described by Warrener et al. (14) was assessed for RNA helicase activity in a standard RNA helicase reaction. Lane 1 represents a sample of the RNA helicase substrate that was boiled prior to electrophoresis to denature the duplex structure and reveal the position in the gel of the release strand product. Lane 2 shows the substrate to which no enzyme had been added. Lane 3 shows the results from a standard helicase reaction containing approximately 200 ng/mL of the YFV NS3 protein.

FIG. 5 is a series of five graphs showing optimal HCV RNA helicase reaction conditions. All data in this figure were generated with the NS3 enzyme derived from bacNS3-5B infected cells using the standard RNA helicase substrate depicted in FIG. 7. Panel A shows the RNA helicase activity in complete reaction mixtures adjusted to various pH values. Panel B shows the RNA helicase activity at or near pH 6.5 using various buffers na-N,N′-bis[2-ethanesulfonic acid; MOPS, 3-[N-morpholino]propanesulfonic acid; KPO₄, potassium phosphate; BIS-TRIS, bis[2-hydroxyethylimino]-tris[hydroxymethyl]methane; TES, N-tris[hydroxymethyl]methyl-2-aminoethane-sulfonic acid; BES, N,N-bis[2-hydroxyethyl]-2aminoethanesulfonic acid; TRIS-acetate, tris(hydroxymethyl) aminomethane acetate; MES,2-[N-morpholino]ethanesulfonic acid; HEPES, N-[2-hydroxyethyl]piperazine-N′-[2-ethanesulfonic acid]; TRIS-HCl, tris(hydroxymethylaminomethane) hydrochloride.

Panel C shows the RNA helicase activity of complete reaction mixtures at pH 6.5 in PIPES buffer and 3 mM MnCl₂ at various ATP concentrations. Panel D shows the RNA helicase activity of complete reaction mixtures at pH 6.5 in PIPES buffer and 1 mM ATP at various MnCl₂ concentrations. Panel E shows the RNA helicase activity of complete reaction mixtures at pH 6.5 in PIPES buffer, 3 mM MnCl₂, and 1 mM ATP at various reaction temperatures.

FIG. 6 is a graphical representation of the RNA helicase activity of the HCV NS3 enzyme derived from bacNS3-5B-infected cells on standard RNA helicase substrate in complete reaction mixtures at pH 6.5 in PIPES buffer, 3 mM MnCl₂, and 1 mM ATP, conducted at 37° C. with three concentrations of the NS3 enzyme. Panel A shows the time course of the reaction at each enzyme concentration. Panel B shows a re-plot of data from panel A to demonstrate the dependence of helicase activity on NS3 enzyme concentration at each reaction time point.

FIG. 7 is a schematic representation of helicase substrates. Shown are duplex nucleic acids used to characterize the substrate specificity of the HCV NS3 helicase. The preparation of these substrates was as described by Warrener and Collett (17). Thick lines represent RNA strands (R), thin lines depict DNA strands (D), and vertical lines represent regions of base pairing. For the DNA-containing substrates, the asterisks denote the radiolabeled release strand. The underlined numbers indicate nucleotide lengths in the base-paired portion of the substrate. Helicase substrates depicted on the right consist of the standard RNA template strand annealed with complementary 22 nucleotide DNA release strands so as to create substrates with no free 3′ unbase-paired nucleotides (22-0), or with 1, 2, 3, or 10 unbase-paired nucleotides to the 3′end of the duplex region (22-1, 22-2, 22-3, and 22-10, respectively).

FIG. 8 presents autoradiographic images of the activity of HCV NS3 RNA helicase on various duplex nucleic acids. The designations and descriptions of helicase substrates are as presented in FIG. 7. Substrates were incubated in standard reaction mixtures and analyzed by electrophoresis on 15% polyacrylamide gels. (A) RNA substrates. (B) DNA-containing substrates. Symbols: Δ substrate boiled prior to electrophoresis; −A, native substrate in standard reaction mixture lacking ATP; +A, complete reaction mixture.

FIG. 9 is a graphical representation of the time course of the HCV NS3 RNA helicase activity on a series of RNA template-DNA release strand substrates that differ in the length on the template strand of their 3′ unbase-paired region. These substrates are schematically depicted in FIG. 7.

FIG. 10 shows an autoradiographic image of 3 microtiter plates in which IC₅₀ curves were generated for anti-viral helicase inhibitors, designated by code numbers 10010 and 10031. These high capacity assays were performed three times in duplicate for the inhibitors; (−) indicates reactions lacking ATP, and (+) indicates complete reactions without inhibitor.

FIG. 11 is a quantitative graphical representation of the data obtained from the high capacity HCV helicase assay shown in FIG. 10. Each individual inhibition curve data set, quantified by beta emission spectrometry, for the six (1 through 6) determinations is plotted for both inhibitor compounds. The concentration of inhibitor necessary to inhibit 50% of the NS3 RNA helicase activity (IC₅₀) is determined by the intersection of the inhibition curve and the horizontal line at 50% control activity.

FIG. 12 shows autoradiographic images of the results of the high capacity HCV NS3 RNA helicase screening assay performed in duplicate on a 96 well plate containing putative anti-viral compounds. When chemically diverse compound collections are screened at compound concentrations of 30 μM, the hit rate is approximately 0.2%.

### DETAILED DESCRIPTION OF THE INVENTION

The Flaviviridae NS3 protein is central to the invention described herein. This protein plays an important role in the life cycle of these viruses catalyzing both gene expression and RNA replication. The NS3 protein is associated with multiple enzymatic activities—in the case of HCV, two distinct proteinases (metalo and serine types), an NTPase, and an RNA helicase—each of which is essential for virus replication. While proteinase and NTPase/RNA helicase activities are located on the same NS3 polypeptide, they are topologically and functionally distinct. The proteinase catalytic domains reside within the amino-terminal one third of the polypeptide, the NTPase/helicase domain is within the carboxy-terminal two-thirds. Mutagenesis of the catalytic site of the serine proteinase, while inactivating that proteinase activity, does not compromise the NS3-associated ATPase activity (34). Moreover, these domains of the NS3 protein can be independently expressed and maintain their respective enzymatic activities (5-7, 9, 10, 12, 14-16, 34-37).

The NTPase/RNA helicase domain of NS3 proteins, are found in members of a large family of proteins, the DEAD/DExH helicases, which include both prokaryotic and eukaryotic cell representatives and numerous virally-encoded polypeptides. Proteins in this family all possess common amino acid sequence motifs that have been associated with (nucleotide triphosphate) NTP binding and hydrolysis activities and with the ability to unwind duplex nucleic acids (38-41). Proteins in this family participate in a variety of biochemical activities involving both DNA and RNA, and include translation, transcription, splicing, recombination, and replication (42, 43).

For positive strand RNA viruses, the putative NTPase/RNA helicase proteins have been subtyped into three groups: alphavirus-like (nsP2-like proteins), picornavirus-like (2C-like proteins), and flavivirus-like (NS3-like proteins) (39, 44).

In the alphavirus-like group, the nsP2 protein of Semliki Forest virus has been shown to have ATPase and GTPase activities (45). From the picornavirus group, the poliovirus 2C protein also exhibits ATPase and GTPase activities (46, 47). RNA helicase activity has yet to be demonstrated for the NTPase/RNA helicase motif-containing proteins from these virus groups.

For the third positive strand RNA virus group of presumed NTPase/RNA helicase proteins, considerable biochemical data are available to substantiate the motif-based predictions of enzymatic activities. RNA-stimulated NTPase activity has been demonstrated for the CI protein of plum pox potyvirus (48), the NS3 protein of the West Nile (11) and yellow fever flaviviruses (14), the NS3 (p80) protein of the pestivirus BVDV (13) and the NS3 protein of hepatitis C virus (12). RNA unwinding (helicase) activity has been demonstrated by Lain et al. (49) for the plum pox potyvirus CI. Warrener and Collett (17) showed the pestivirus BVDV NS3 protein is likewise an RNA helicase. The present invention discloses the yellow fever virus (YFV) NS3 protein has RNA helicase activity, and finally, Kim et al. (15), Jin and Peterson (16), and the present invention show that the HCV NS3 protein is an RNA helicase.

The present invention is directed to the identification and development of agents capable of inhibiting infectious processes associated with certain RNA viruses. In accordance with the present invention, there are provided the following: methods and processes for producing and preparing reagents suitable for use in a biochemical assay for critical enzyme activities associated with human hepatitis C virus and related viruses; methods and processes for biochemical assays suitable for use in screening for and discovering drugs to treat infections and diseases associated with human hepatitis C virus and related viruses, and the use of said materials, methods, processes, and assays for the discovery of antiviral compounds to treat infections and diseases associated with these viruses. Preferred procedures for the expression and purification of an enzymologically active NS3 protein, in particular the NS3 protein's NTPase/RNA helicase activities, are disclosed. In addition, preferred enzymological reaction conditions for the NS3 protein NTPase/RNA helicase are provided. Furthermore, the invention provides an assay for the high capacity measurement of the NS3 protein RNA helicase activity, and also teaches the use of both NS3 protein and high capacity assay for purposes of screening agents potentially capable of inhibiting the enzyme's NTPase activity or its RNA helicase activity.

The examples set forth below are provided to describe the invention in greater detail. They are not intended to limit the invention.

### EXAMPLE 1

### Expression of the HCV NS3 Protein

Since HCV can not be propagated efficiently in vitro it is necessary to obtain the viral genetic material from tissues or fluids of infected humans or chimpanzees using techniques of molecular biology known to those skilled in the art. These include, but are not limited to the synthesis of complementary DNA (cDNA) with reverse transcriptase followed by gene amplification using the polymerase chain reaction (PCR) as set forth in Current Protocols in Molecular Biology, Ausubel et al., eds., John Wiley & Sons, Inc. (1995). The products of these reactions are double stranded DNA copies of the viral RNA genome inserted into standard plasmid vectors. As such, these molecular clones of the viral genome are readily manipulated by standard molecular, biologic and genetic engineering techniques. Numerous molecular clones of HCV have been so generated by many groups working in the field. One such clone, derived from HCV strain H was provided by Dr. Charles M. Rice of Washington University, St. Louis under a licensing agreement with that University.

The NS3 gene represented in molecular clones of the HCV genome may be engineered into a number of recombinant DNA expression systems for the production of its encoded protein. These systems include, but are not limited to those utilizing bacteria (e.g., E. coli, B. subtilis, and others), fungi (e.g., S. cerevisiae, P. pastoris, and others), and plant, insect, and mammalian cells. These systems may employ transient transfection procedures utilizing recombinant viral vectors such as baculoviruses or vaccinia viruses or stable transfection methods using dominant selectable markers. All of these systems, and variations upon them, are available to, or are readily generated by one of ordinary skill in the art of molecular biology and genetic engineering.

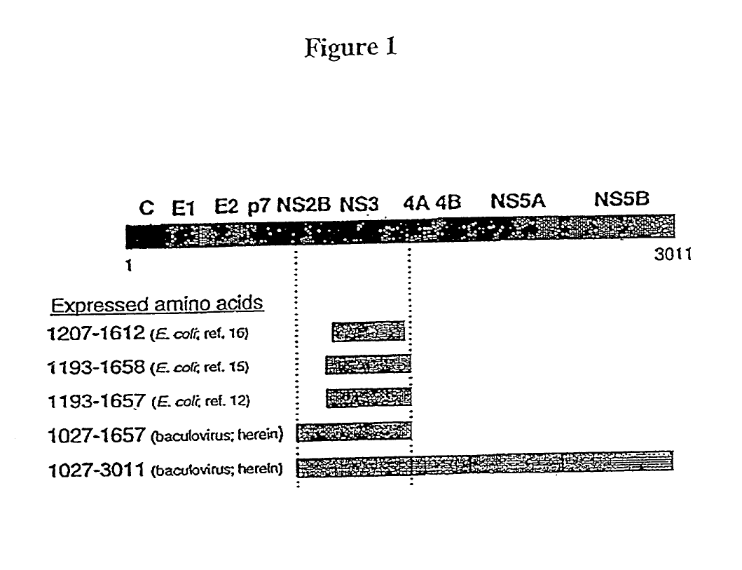

Furthermore, the NS3 gene may be engineered so as to express the complete coding sequence of the NS3 protein, truncated or modified versions of the complete coding sequence, or in conjunction or combination with other amino acid or polypeptide coding elements. Within the approximately 3011 amino acids of the open reading frame of HCV, the NS3 protein is coded for by residues 1027-1657 (these precise coordinates vary slightly among HCV isolates) (FIG. 1). Thus, in one study, the carboxy-terminal portion of the NS3 protein derived from HCV encoding 464 amino acids (residues 1193-1657) may be expressed in E. coli as taught by Suzich et al. (12). Kim et al. (15) expressed a very similar portion of the HCV NS3 gene (residues 1193-1658) to which they appended 6 non-viral histidine residues in order to facilitate the subsequent purification of the resultant polypeptide by metal-binding chromatography. Jin and Peterson (16) expressed in E. coli, a 406 amino acid carboxy terminal portion of the NS3 gene (amino acids 1207-1612) to which they also appended a polyhistidine tag. As with HCV, the comparable carboxy-terminal portion of the NS3 protein derived from the YF flavivirus encoding 460 amino acids may be expressed in E. coli as taught by Warrener et al. (14).

All of the above-described modified (amino-terminal truncated) NS3 proteins possessed detectable NTPase activity and RNA helicase activity. However, in all cases, the recombinant proteins produced did not represent a complete and authentic sequence of the natural NS3 protein. In the instant invention, the complete and authentic sequence of the HCV NS3 protein has been engineered in a recombinant baculovirus expression system. This was performed using two methods. In one method, the NS3 gene was amplified from a molecular clone (obtained from Washington University) in such a fashion that a single methionine codon (ATG) was added immediately upstream of the first amino acid residue of the natural NS3 protein (residue 1027) and a translational stop codon (TAA) was added immediately following the last amino acid residue of the protein (residue 1657; FIG. 1, bacNS3). In an alternative method, the nonstructural protein coding region beginning with the NS3 protein (residue 1027) and extending to the natural viral termination codon beyond the last residue of the open reading frame (residue 3011), was engineered (FIG. 1, bacNS3-5B). Each of the NS3 gene-containing elements were inserted into a standard baculovirus transfer vector (pVL1393; ref. 50). The subsequent generation of the respective recombinant baculoviruses was carried out by standard procedures (51). Infection of Spodoptera frugiperda Sf9 cells with the so produced bacNS3 and the bacNS3-5B recombinant baculoviruses resulted in the production of the expected mature HCV proteins. In the case of bacNS3, the 70 kDal NS3 protein was produced as detected by either radioimmunoprecipitation or Western immunoblotting techniques using NS3 specific antibodies. Antibodies against viral proteins can be generated using standard techniques known in the art, these may include both polyclonal and monoclonal antibody preparations. Expression of the bacNS3-5B recombinant virus resulted in the detection of the mature, naturally processed NS3, NS4A, NS4B, NS5A, and NS5B HCV proteins in lysates from infected insect cells.

A preferred embodiment of the invention involves: i) expression of a full length, authentic NS3 polypeptide such as in recombinant baculoviruses, such as bacNS3 and bacNS3-5B, and ii) expression in a eukaryotic system such as insect cells as described above.

### EXAMPLE 2

### Purification of the HCV NS3 Protein

For use in assays to identify anti-viral agents, the genetically engineered NS3 proteins produced as described above must be isolated and purified in soluble form. An engineered expression system may have as part of its design, aspects that allow the secretion or excretion of the desired protein from the cell, thus facilitating purification of the protein from the cell culture medium. Alternatively, expressed intracellular protein may be released upon disruption or dissolution of the cell. Cellular disruption may be effected by any number of procedures involving both mechanical or chemical means, all of which are familiar to those skilled in the art of protein biochemistry and protein purification. The procedures to be utilized in a particular case, are those in which proteins are maintained in their native conformation so as to retain their full and natural characteristics and enzymological activities. However, in some cases, non-native proteins may be renatured as a part of the process of protein extraction from cells. Renaturation of proteins is often relevant with material expressed in procaryotic systems (e.g., E. coli). For example, Suzich et al. (12) demonstrate that a version of the HCV NS3 protein expressed in E. coli appears as a denatured aggregate, and that this aggregate may be denatured and renatured to yield enzymologically active protein.

In a preferred embodiment of the invention, proteins expressed in their native state are extracted from cells in a manner that maintains the native state of the protein. Extraction may be performed with mechanical methods such as gentle shearing of cells in hypotonic buffers or decavitation, or by chemical methods employing solvents compatible with the maintenance of the natural structure and activity of enzymes. For example, any number of nonionic detergents (e.g., Triton X-100, NP-40, and others), ionic detergents (deoxycholate, cholate, and others), or various combinations of ionic and/or nonionic detergents may be used. In one embodiment of the invention, RIPA buffer ((0.15 M NaCl/10 mM Tris-HCl, pH 7.2/1.0% Triton X-100/1.0% deoxycholate/0.1% sodium dodecyl sulphate/1 mM EDTA) is useful for the dissolution of eukaryotic cells expressing native forms of the NS3 protein intracellularly.

Following removal of insoluble material and debris either by filtration or centrifugation, the soluble protein extract is then subjected to protein enrichment and purification procedures. Any number of recognized protein purification procedures may be employed singly and in any combination to obtain preparations enriched for the NS3 protein.

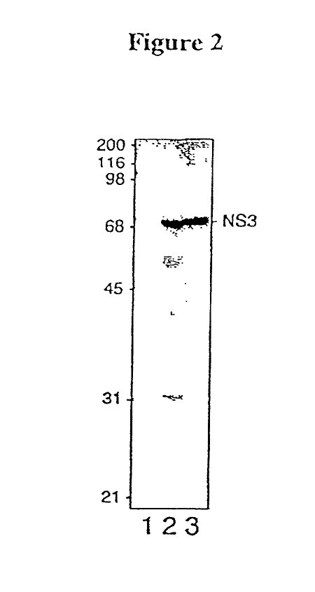

In a preferred embodiment of this invention, immunoaffinity chromatography may be used to enrich for the NS3 protein from cellular extracts. General procedures for immunoaffinity chromatography are described by Erikson et al. (52). More specifically, antisera derived from either humans or chimpanzees infected with HCV, or alternatively antisera specifically generated by immunization of suitable hosts with immunogens containing immunologic determinants of the NS3 protein may be used. In any case, the preferred antiserum for use in immunoaffinity purification of NS3 protein should interact with the native or natural NS3 protein. Sera from HCV-positive plasma donors were initially screened for the presence of antibodies reactive with NS3 protein produced in recombinant baculovirus bacNS3-5B-infected Sf9 cells by radioimmunoprecipitation. Sera with the greatest reactivity and selectivity for this native protein were selected for further use. Sera were pooled from 12 donors and used to prepare immunoglobulin (IgG) by standard protein G chromatography. IgG (50 mg) was coupled to 2 mL of Affi-Gel 10 resin (BioRad) according to the manufacture's instructions. Frozen baculovirus-infected Sf9 cell pellets containing approximately 10×10⁷ infected cells were lysed in RIPA buffer supplemented with protease inhibitors (final concentrations: 1 mM phenylmethylsulfonyl fluoride, 5 μg/mL leupeptin, 50 μg/mL antipain and 1 μg/mL pepstatin). Lysates were cleared by centrifugation at 100,000× g for 30 minutes and were then applied to the antibody column at a flow rate of 10 mL/hr. The column was washed with 6 column volumes each of RIPA buffer with protease inhibitors, STE buffer (150 mM NaCl/10 mM Tris-HCl, pH 7.2/1 mM EDTA), 1M buffer (1 M NaCl/10 mM Tris-HCl, pH 7.2/1 mM EDTA/0.1% NP-40), EG buffer (40% ethylene glycol/1 M NaCl/10 mM Tris-HCl, pH 7.2/1 mM EDTA), and STE buffer. Bound proteins were eluted with 2 M KSCN buffer (2 M KSCN/10 mM Tris-HCl, pH 8.0/1 mM EDTA). Protein-containing fractions were pooled and dialyzed against 50% glycerol buffer (50% glycerol/50 mM NaCl/10 mM Tris-HCl, pH 7.2/1 mM EDTA/0.01% 2-mercaptoethanol/0.01% Triton X-100) and stored at −20° C. FIG. 2 shows the enriched NS3 protein preparations derived from bacNS3-infected and bacNS3-SB-infected insect cells.

### EXAMPLE 3

### NTPase and RNA Helicase Activity of Purified Full Length HCV NS3 Protein

The NTPase and RNA helicase activities of the HCV NS3 protein have been previously disclosed by Suzich et al. (12), Kim et al. (15) and Jin and Peterson (16). In all these examples, the NS3 protein was produced in E. coli and represented variously modified versions of the authentic polypeptide. For example, Suzich et al. describe an amino-terminal truncated version of the protein representing amino acid 1193 through 1657 of the authentic protein. Kim et al. similarly expressed residues 1193-1658 of the NS3 protein to which 6 non-viral histidine residues were appended. Jin and Peterson (16) also added a polyhistidine tag to amino acids 1207-1612 of the NS3 protein. The complete NS3 protein coding region encompasses residues 1027-1657 (FIG. 1).

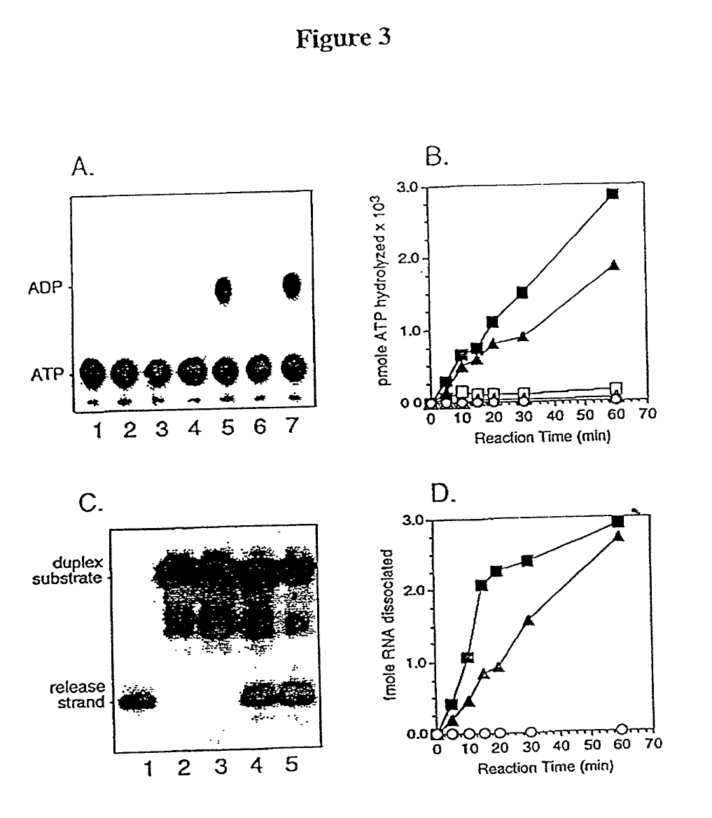

In a preferred embodiment of the invention, the NTPase and RNA helicase activities of the NS3 protein are derived from the full length, native polypeptide (residues 1027-1657). The ATPase and helicase activities associated with this version of the HCV NS3 protein, purified from either recombinant baculovirus bacNS3-infected or bacNS3-5B-infected insect cells as described in Example 2 above, are illustrated in FIG. 3. As previously shown for the truncated versions of the NS3 protein (12, 15, 16), the ATPase activity associated with the full length proteins disclosed herein is stimulated by addition to the reaction of polynucleotides (FIGS. 3A & 3B).

Following purification of an enzyme from a genetically engineered expression system, it is necessary to optimize reaction conditions to obtain maximum enzyme efficiency. By performing standard enzymologic kinetic analyses, the catalytic activities of these purified proteins were evaluated. Quantitative measurement of the conversion of substrate to product by a known amount of enzyme per unit time allows an assessment of the turnover rate and an estimate of the catalytic efficiency of the enzyme. Thus, the rates of ATP hydrolysis (NTPase activity) and of RNA strand displacement (RNA helicase activity) can be measured and used to compare enzyme preparations. The results of such analyses for the NTPase and RNA helicase activities of various NS3 proteins is provided in Table 1. Also included in the table are available data relating to the catalytic efficiency of the amino-terminal truncated NS3 proteins disclosed by Kim et al. (15), Jin and Peterson (16), and Suzich et al. (12). It is to be noted that the basal level of ATPase, that is, the ATPase activity in the absence of polynucleotide, of the presently disclosed forms of the NS3 enzyme is considerably lower than those previously disclosed for the various truncated versions of the NS3 protein (12, 15, 16). This difference may reflect some inherent enzymologic difference between full length and truncated versions of the NS3 protein, some characteristic of material obtained from E. coli, the presence of contaminant materials or activities, or differences resulting from the means of protein purification. Regardless, this fundamental difference serves to distinguish the present enzymes from its predecessors. Additionally, the RNA helicase catalytic activity of the full length NS3 protein is significantly higher (10-25 fold) than truncated or modified versions of the enzyme.

Furthermore, comparison of the kinetics of the RNA helicase activity of the full length authentic NS3 protein derived from bacNS3-infected cells with those of the NS3 protein derived from bacNS3-5B-infected cells suggests that the latter has greater activity (FIG. 3C, Table 1). Possible explanations for this result include the presence in the latter NS3 protein preparation of a factor that enhances the NS3 RNA helicase activity. This putative enhancer may be another protein, either of host cell or HCV origin. In particular, it may be that other HCV nonstructural proteins expressed in bacNS3-5B-infected cells co-purify with the NS3 protein and associate or interact with the NS3 protein to improve the catalytic efficiency of the enzyme. Thus, a preferred embodiment of the invention uses HCV NS3 RNA helicase activity derived from cells expressing the NS3 through NS5B region of the HCV genome.

TABLE 1
ATPase and RNA Helicase Catalytic Rates
for Different Versions of the HCV NS3 Protein

| | | Catalytic Activity (min⁻¹) | | |
|---|---|---|---|---|
| | NS3 | non-authentic | ATPase | RNA |
| NS3 Protein | amino acids | amino acids | (−/+ poly U) | Helicase |
| ntNS3 (ref. 16) | 1207-1612 | yes | 400/470 | NR |
| ntNS3 (ref. 15) | 1193-1658 | yes | NR | NR |
| ntNS3 (ref. 12) | 1193-1657 | yes | 200/1300 | <0.0004* |
| bacNS3 | 1027-1657 | no | 0/659 | 0.003 |
| bacNS3-5B | 1027-1657 | no | 23/1046 | 0.01 |

Notes: −/+ poly U indicates ATPase activity in the absence or presence of poly(U);
NR = not reported;
*based on unpublished results of authors of ref. 12.

### EXAMPLE 4

### NTPase and RNA Helicase Activity of the Pestivirus NS3 Protein

For the related pestivirus BVDV NS3 protein (p80 protein), the NTPase and RNA helicase activity of the full length, authentic polypeptide produced in a baculovirus-insect cell system has been disclosed by Tamura et al. (13) and Warrener and Collett (17). These disclosures show that the activities associated with the pestivirus NS3 protein are similar to those described herein for the HCV NS3 protein.

### EXAMPLE 5

### NTPase and RNA Helicase Activity of the Flavivirus NS3 Protein

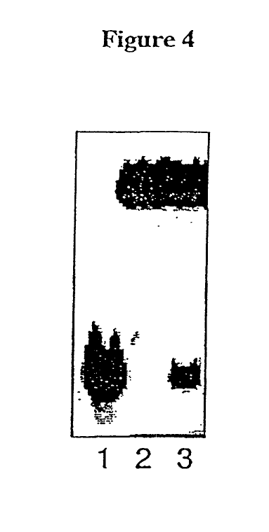

For the flavivirus NS3 protein, the NTPase activity derived from a proteolytic fragment of the West Nile flavivirus NS3 protein representing the carboxy terminal portion of the protein was disclosed by Wengler and Wengler (11). Warrener et al. (14) expressed an amino-terminal truncated version of the YFV NS3 protein in E. coli. This recombinant NS3 protein so produced possessed NTPase activity (14) and RNA helicase activity (FIG. 4).

### EXAMPLE 6

### Characterization and Optimization of the HCV NS3 RNA Helicase Activity

From the examples set forth above, it is clear that the full length, native HCV NS3 protein possesses superior enzymologic characteristics relative to modified versions of the same polypeptide. Enzyme prepared by these methods provides great utility for the development of effective anti-viral agents. A preferred embodiment of the present invention envisions that a full length, authentic sequence, native virus-encoded protein possessing the described NTPase and RNA helicase activities be used for these purposes.

For such proteins to be optimally employed, it is desirable that the methods utilized for the measurement of the enzymatic activities be amenable to the ready evaluation of large numbers of test samples. Thus, development of a high capacity or high throughput assay for such enzymatic activities is required to facilitate the identification of effective anti-viral drugs. For example, to develop a high throughput assay for measurement of HCV NS3 RNA helicase activity, it is necessary to understand the optimal reaction conditions and kinetic parameters of the enzyme. To this end, immunoaffinity purified recombinant full length NS3 protein derived from bacNS3-5B-infected insect cells was used to characterize and optimize the RNA helicase activity with respect to: a) reaction conditions; b) enzyme kinetics; and c) substrate specificity. The methods used are set forth below.

a. Reaction Condition Optimization.

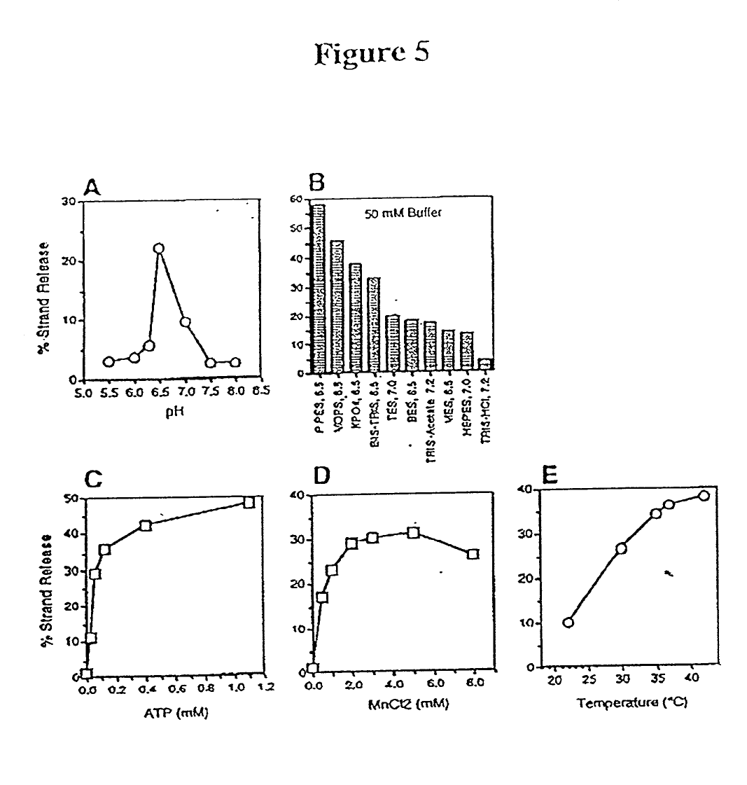

Several parameters were systematically investigated. Reaction pH was assessed from pH 5.5 to 8.0. Maximal activity was observed at pH 6.5 (FIG. 5A). The reaction buffer used to maintain this pH was also assessed. PIPES buffer at pH 6.5 is preferred (FIG. 5B). Divalent cations are required for activity. Both Mn⁺² and Mg⁺² supported comparable levels of activity over a broad concentration range (2 to 8 mM; shown for Mn⁺² in FIG. 5D). When tested at 3 mM, Zn⁺², Ca⁺², and Cu⁺² were able to substitute for Mn⁺², but with activity levels reduced 2-, 3-, and 7-fold, respectively. The monovalent cations Na⁺ and K⁺ inhibit HCV NS3 helicase activity. The HCV RNA helicase activity was strictly dependent on the presence of NTP. When tested individually at 1 mM, all eight of the common NTPs supported the helicase activity at similar levels. Helicase activity over a range of ATP concentrations (FIG. 5C), followed by Lineweaver-Burke analysis, indicated a Kₘ for ATP of 50 μM. With the optimal reaction conditions and the standard RNA substrate (see below), helicase activity was assessed at various reaction temperatures. Activity increased up to 35° C. and then leveled off (FIG. 5E).

b) Enzyme Kinetics.

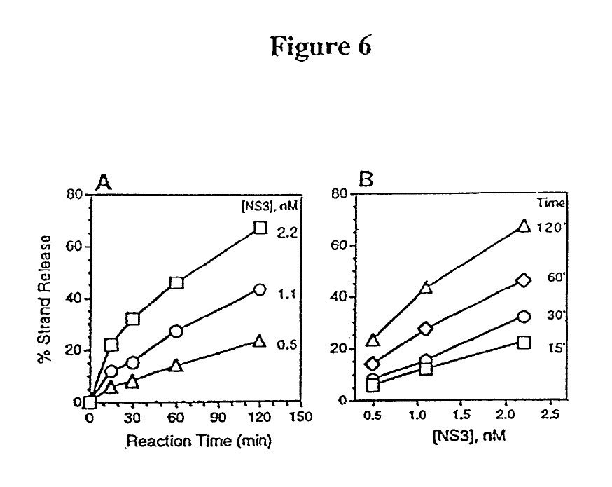

The rate of helicase strand displacement with respect to time and enzyme concentration was examined (FIG. 6). The kinetic parameters Kₘ and k_{cat}, (turnover number) were determined for the standard RNA substrate. Evaluation of enzymatic activity at varying concentrations of the RNA substrate, followed by Lineweaver-Burke analysis of the data, revealed a Kₘ for RNA substrate of 0.5 nM and a turnover number of 0.01 pmol/min/pmole enzyme at 200 ATP.

c) Substrate Specificity.

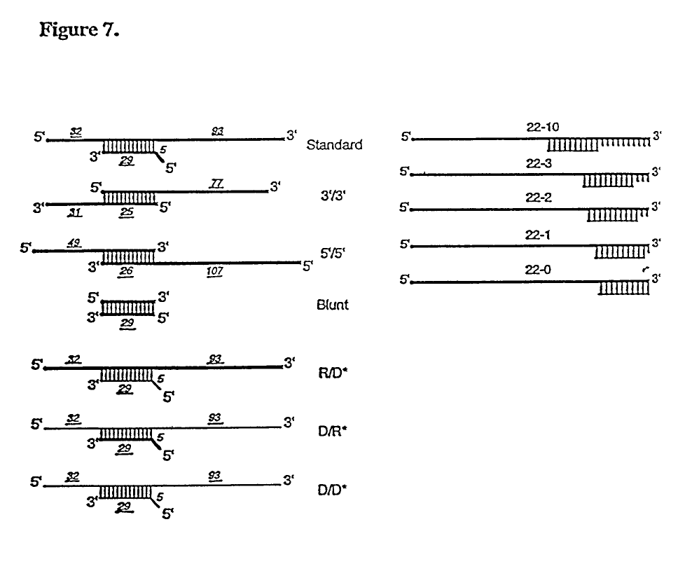

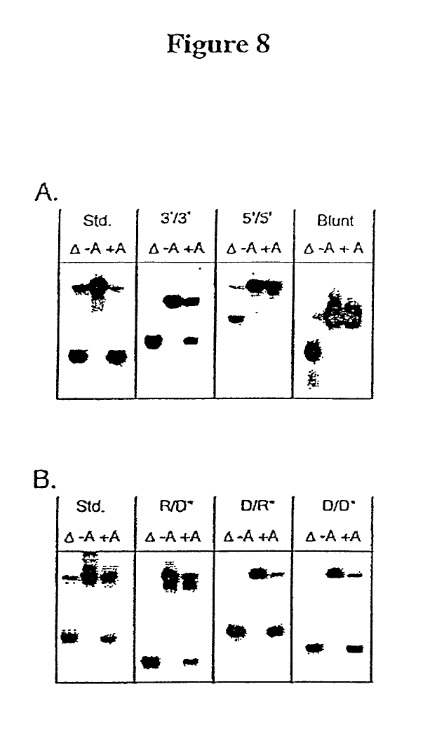

To determine the substrate specificity of the HCV NS3 enzyme, we investigated the ability of the helicase to act on a variety of nucleic acid substrates. RNA substrates (prepared as described in reference 17) containing only 3′ single strand regions (3′/3′), only 5′ single strand regions (5′/5′) and no single strand regions (blunt-end RNA) were constructed along with the standard RNA substrate which contains both 3′ and 5′ single strand regions (FIG. 7). Substrates containing a 3′ single strand region (FIG. 8A, Std. and 3′/3′) were acted on by the NS3 helicase. However, the 5′/5′ and blunt-end substrates were not utilized by the enzyme (FIG. 8A). These results are indicative of an RNA helicase with a 3′-to-5′ directionality of strand dissociation with respect to the template strand. This is the same directionality that was observed previously for the pestivirus NS3 helicase enzyme (17).

The ability of the NS3 helicase to utilize DNA/RNA, RNA/DNA and DNA/DNA substrates was also examined (FIG. 8B). The NS3 enzyme efficiently dissociated the “standard” substrate in which the RNA template strand was replaced with a DNA strand of identical sequence (D/R*), or in which the RNA release strand was replaced with a DNA strand of identical sequence (R/D*) (FIG. 8B). The enzyme also was able to dissociate the strands of a DNA/DNA substrate (D/D*) (FIG. 8B).

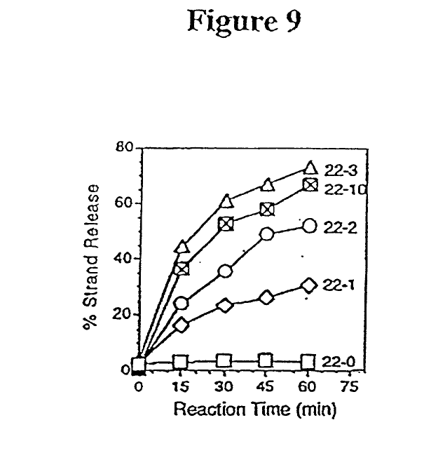

Since the HCV helicase appeared to require a duplex substrate with a 3′ single strand region, this was investigated further. To determine the minimum length of the 3′ single strand region necessary for activity, we constructed RNA/DNA substrates containing 3′ unbase-paired regions on the template strand of 0, 1, 2, 3, and 10 nucleotides in length (22-0. 22-1, 22-2, 22-3, and 22-10, respectively) (FIG. 7). As shown in FIG. 9, the helicase does not act on 22-0, consistent with the lack of activity on the blunt ended substrate shown in FIG. 8A. However, the helicase utilized a substrate with only a single unbase-paired nucleotide. However, both the rate and extent of strand dissociation were diminished with substrates 22-1 and 22-2 relative to those substrates with 3 or 10 unbase-paired nucleotides, where full activity was achieved.

Based on the above substrate specificity data, and in view of the fact that the HCV helicase in its authentic role acts only on RNA in the context of the virus replication complex, the “standard” RNA duplex substrate is a preferred substrate for use in the evaluation of HCV RNA helicase activity.

### EXAMPLE 7

### High Throughput Assay for Measurement of HCV NS3 RNA Helicase

There are numerous methods available for the measurement of the HCV NS3 RNA helicase activity. For example, in a gel electrophoresis method in which the release strand of a duplex helicase substrate is detectably labeled, e.g., with a suitable radioisotope such as ³²P, the release strand product may be resolved from the unreacted substrate by electrophoresis of the terminated reaction mixture on a polyacrylamide gel (for example, see FIGS. 3B and 8). The amount of radioactivity associated with the release strand and unreacted substrate are directly quantified by phosphorimaging, or any other suitable methodology, and the percent strand displacement is calculated. While this assay format may be used to evaluate and screen compounds for inhibition of the RNA helicase activity, it is quite laborious. Another analytical method for the measurement of activity uses a continuous fluorescence-based assay to monitor helicase-catalyzed strand displacement (53-55). Additional methods for the measurement of helicase activity exist or can be envisaged.

There are numerous approaches for a high capacity RNA helicase screening assay that are considered to be within the scope of this invention. Several approaches require that the RNA substrate be modified by any number of means. In one example, the template RNA strands may be synthesized in the presence of biotin-21-UTP to which a ³²P-labeled release strand is annealed. This duplex substrate is subsequently bound to a streptavidin-coated 96-well plate, and the emission of radioactivity from the plates is measured. However, it has been found that immobilized RNA substrates behave poorly and exhibit altered reaction kinetics relative to substrates in solution. Furthermore, the modification, in any form, of the RNA substrate presents the possibility that such modification (e.g., the presence of a biotin moiety on the RNA) might affect the enzymology in some manner, and in turn, compromise one's ability to identify effective inhibitors. Analytical methods involving modified substrates are least preferred in this invention.

Another example of an approach to development of a high capacity assay for the HCV RNA helicase makes use of Amersham's scintillation proximity technology assay (SPA). Amersham offers a DNA helicase assay kit based on the SPA (56). While this technology can be applied to HCV RNA helicase, the assay is insensitive. It requires 50 times more enzyme and RNA substrate per assay point than that required in the instant invention, to achieve acceptable signals, and is significantly more expensive than the preferred assay system which is described below.

Several criteria must be considered when developing a useful high capacity assay system including: throughput, quantitative accuracy and reproducibility, acceptable signal-to-noise ratio, efficient use of reagents, and suitability for automation. All of these criteria are satisfied by the following method. Helicase reactions using ³²P-labeled standard (unmodified) RNA substrate (FIG. 7) are carried out in 96-well plates. After a suitable incubation period, reactions are terminated by addition of a stop/capture solution containing a biotinylated DNA oligonucleotide complementary to the RNA release strand. After an annealing period, the RNA release strand/DNA hybrids are quantitatively captured onto streptavidin-coated agarose beads (Pierce). The beads are then collected onto filter paper, and the associated radioactivity is quantified on a phosphorimager (Molecular Dynamics) or other similar devices. Many of the steps in the assay have been automated on the Biomek 2000 Laboratory Workstation (Beckman Instruments). Additional features involved in the development of the preferred high throughput assay are described in greater detail below.

a) Substrate Preparation.

The template RNA strand was transcribed from the SP6 promoter of a BstN1-digested pSP6 plasmid (Promega). The release strand was transcribed from the SP6 promoter of a BamH1-digested pSP64 plasmid (Promega) in the presence of α³²P-CTP (Amersham). In selecting a detectable label for the helicase substrate release strand, the RNA was maintained in as natural a state as possible and the use of modified NTPs (e.g. ³⁵S-[thio]-CTP) was avoided. The two RNA transcripts were hybridized, and the RNA/RNA duplex was purified by polyacrylamide gel electrophoresis to yield the standard substrate.

b) Capture System

Any suitable capture system may be utilized in practicing the method for assying a compound for anti-viral activity against hepatitis C virus in accordance with the present invention. A preferred capture system comprises a specific binding pair, one member of the specific binding pair being conjugated with an oligonucleotide having a nucleotide sequence complementary to the detectably labeled release strand and the other member of the specific binding pair being affixed to a solid support. Following capture, the detectable label present in the release strand, is quantitated as a measure of the anti-viral activity of the putative anti-viral compound.

b-i) Biotinvlated DNA Oligomer for Capture of Free RNA Release Strand.

A DNA oligomer complementary to the RNA release strand and modified at its 5′ terminus with biotin was prepared commercially (Pierce, Rockford, Ill.). Three aspects of the biotinylated DNA oligomer capture approach are deemed critical to the success of the assay format: i) verification that hybridization of the biotinylated DNA oligomer to the RNA release strand was quantitative; ii) confirmation that the presence of the DNA oligomer did not disrupt unreacted substrate; and iii) assurance that the presence of the DNA oligomer prevented back hybridization of the RNA release strand to free RNA template strand.

To determine the optimal conditions for capture of free release strand by the biotinylated DNA oligomer, an amount of substrate representing twice that used in the standard helicase reaction was heat denatured, quenched on ice, and diluted with hybridization buffer. The kinetics of biotinylated DNA oligomer capture of free RNA release strand were examined over a range of oligomer-to-release strand molar ratios (from 0:1 to 160:1). Capture was monitored by gel shift of the ³²P-labeled release strand on nondenaturing polyacrylamide gels. Gel shift assay methods are described in greater detail in Current Protocols in Molecular Biology, Ausubel et al., eds., John Wiley & Sons, Inc. (1995). RNA release strand capture was complete within 20 minutes at a 20:1 molar ratio of biotinylated oligomer-to-release strand. Duplex RNA substrate did not undergo any detectable strand separation, nor was back hybridization apparent under the capture conditions used. To provide a further margin for the completeness of this important capture step, a ratio of DNA oligomer-to-RNA release strand of 80:1 and a capture time of 45 minutes was chosen for the preferred high capacity assay.

b-ii) Capture of Biotinylated DNA Oligomer/RNA Release Strand Hybrid on Streptavidin-coated Aqarose Beads.

Streptavidin-coated agarose beads (Pierce) were used to harvest biotinylated DNA oligomer/RNA release strand hybrids. The kinetics of bead capture of the hybrids were investigated by incubating a known amount of hybrid with varying amounts of beads, and then measuring the quantity of radioactivity associated with the beads over time. A 1:100 dilution of the streptavidin-coated beads was sufficient to capture all hybrids in 30 minutes. To ensure complete capture in the screening assay, a 1:100 dilution of coated beads was co-incubated with the biotinylated hybrids for 60 minutes.

b-iii) Streptavidin Bead Collection on Filter Paper.

After capture of the biotinylated DNA oligomer/RNA release strand hybrid on beads, the beads were collected onto filter paper in a 96-well vacuum manifold. The beads were washed extensively to remove unreacted substrate RNA. The manifold was then disassembled to expose the filter paper while maintaining the vacuum, and a single sheet of pressure sensitive film was applied over the filter. The film was laminated onto the filter by brief heating under vacuum. Radioactivity remaining on the filter paper was then quantitated by phosphorimaging.

### EXAMPLE 8

### Validation of the High Capacity Assay for Helicase Activity

Assay validation is necessary to ensure that the assay is reproducible, reliable, and that it provides the greatest opportunity for identifying inhibitors. Several validation experiments are performed to minimize false positives, control variability, and provide accurate quantitation of compound potency. This latter point is especially important during medicinal chemistry structure activity relationship (SAR) studies used in the identification and development of antiviral agents.

a) Enzyme Qualification.

Each new preparation of NS3 enzyme is quantified and assessed for purity by SDS-polyacrylamide gel electrophoresis. The RNA helicase activity is determined over a time course at several enzyme concentrations, and the rate of strand displacement is calculated. This value must fall within 20% of the previously described turnover number to be qualified for the high throughput assay.

b) Substrate Qualification.

The radiolabeled double stranded RNA substrate is quantified by ethidium bromide or Syber green fluorescence using tRNA standards and scintillation spectrometry. To qualify for use in the screening assay, the specific activity of the substrate should be between 1-10×10⁴ cpm/ng. Additionally, the RNA substrate is tested in the helicase gel migration assay at several concentrations to verify the quality of the RNA and extent of strand dissociation

c) Reaction Conditions.

The preferred screening assay reaction conditions are designed to provide a high level of sensitivity for enzyme activity inhibition and allow for identification of inhibitors of either the enzyme's ATPase or helicase activities. These conditions were adjusted to be at or near the Km values for enzyme substrates (RNA and ATP) and to be within the linear portion of the activity curve with respect to both the reaction time and enzyme concentration. Reaction conditions may be conducted at 30-42° C. for 10-120 minutes in a solution containing 10-100 mM PIPES buffer, pH 6.0-7.5, 0.5-2 mM dithiothreitol or 2-mercaptoethanol, 1-200 g/mL bovine serum albumin, 0.5-8 mM MnCl₂ or MgCl₂, 0.025-5 mM ATP, 0.2-2 U/mL RNasin or other suitable RNase inhibitor, 3-3000 ng/mL RNA substrate, and 6-6000 ng/mL NS3 enzyme. In a preferred embodiment of the invention, reactions are conducted at 37° C. for 45 minutes in a solution containing 50 mM PIPES buffer, pH 6.5, 1 mM dithiothreitol, 100 g/mL bovine serum albumin, 3 mM MnCl₂, 0.2 mM ATP, 1 U/mL RNasin, 30 ng/mL RNA substrate, and 60 ng/mL NS3 enzyme.

d) High Capacity Assay.

The attributes of the preferred high capacity assay, which is suitable for the identification of inhibitors of NTPase and RNA helicase activities of HCV and related viruses, are as follows:

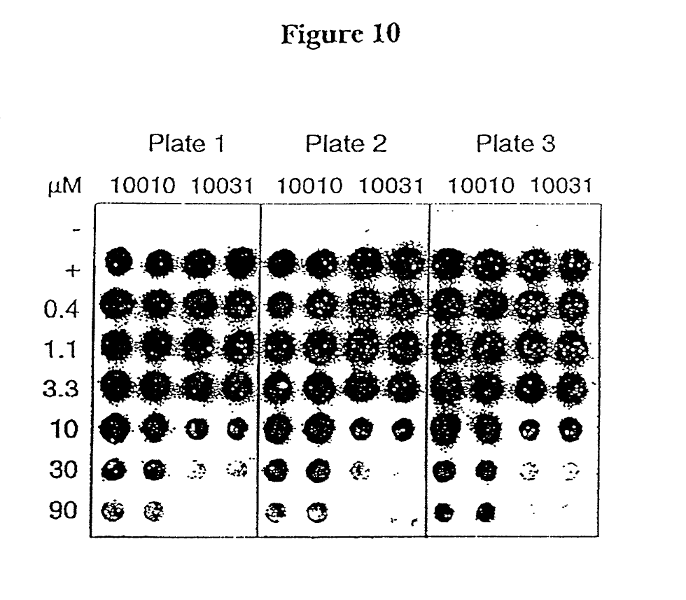

i) Consistent and acceptable signal-to-noise ratio of 8-12 (FIG. 10, row (+) vs. row (−)).

ii) Reproducible.

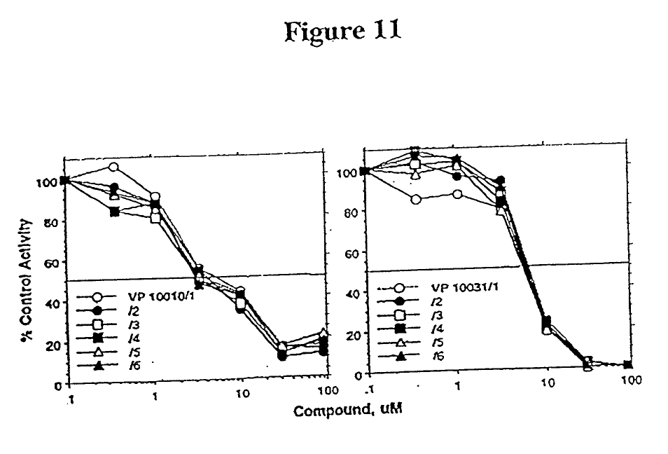

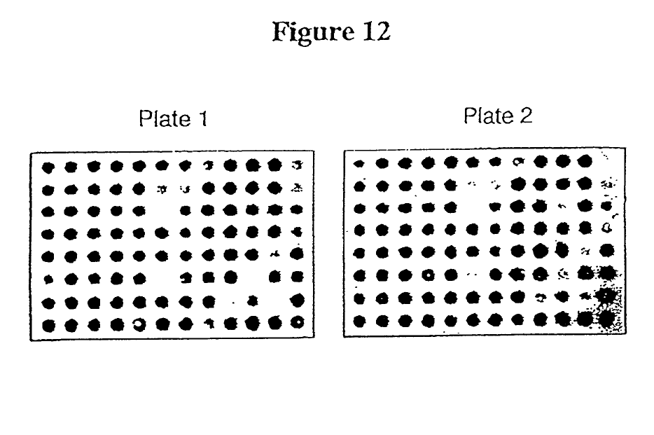

Identical reactions repeatedly yield quantitatively similar results (FIGS. 10-12). An autoradiograph of the assay used to generate IC₅₀ data sets for the two reference helicase inhibitors, noted above, which are proprietary products of the assignee of the present invention, is presented in FIG. 10. The quantitative values (% of control strand displacement) from the six individual data sets derived from three different assay plates are plotted in FIG. 11. IC₅₀ values from each data set were nearly identical, and furthermore, were identical to values obtained in the gel assay. Well-to-well and plate-to-plate variability are sufficiently low such that only duplicate reactions need be run.

iii) High Throughput.

The throughput of the assay in its current configuration is approximately twelve 96 well plates/day/person.

Certain preferred embodiments of the invention have been described and exemplified herein. However, other embodiments will be apparent to persons skilled in the art. Thus, the invention is not limited to the embodiments specifically described, but may be varied and modified within the scope of the appended claims.

### REFERENCES

1. Choo Q. L., Kuo, G., Weiner, A. J., Overby, L. R., Bradley, D. W., and Houghton, M. (1989) Science 244:359-362.

2. Murphy, F. A., Fauquet, C. M., Bishop, D. H. L., Ghabrial, S. A., Jarvis, A. W., Martelli, G. P., Mayo, M. A., Summers, M. D. (1995) Virus Taxonomy. Springer-Verlag, Wien New York.

3. Muerhoff, S. A., Leary, T. P., Simons, J. N., Pilot-Matias, T. J., Dawson, G. J., Erker, J. C., Chalmers, M. L., Schlauder, G. G., Desai, S. M., Mushahwar, I. K. (1995) J. Virol. 69:5621-5630.

4. Foung, S, personal communication, 1995.

5. Bartenschlager, R., Ahlborn-Laake, L., Mous, J., Jacobsen, H. (1993) J. Virol. 67:3835-3844.

6. Eckart, M. R., Selby, M., Masiarz, F., Lee, C., Berger, K., Crawford, K., Kuo, C., Kuo, G., Houghton, M., Choo, Q-L. (1993) Biochem. Biophys. Res. Comm. 192:399406.

7. Grakoui, A., McCourt, D. W., Wychowski, C., Feinstone, S. M., Rice, C. M. (1993) J. Virol. 67:2832-2843.

8. Grakoui, A., McCourt, D. W., Wychowski, C., Feinstone, S. M., Rice C M. (1993) Proc. Natl. Acad. Sci. USA 90: 10583-10587.

9. Hijikata, M., Mizushima, H., Akagi, T., Mori, S., Kakiuchi, N., Kato, N., Tanaka, T., Kimura, K., Shimotohno, K. (1993) J. Virol. 67:4665-4675.

10. Tomei, L., Failla, C., Santolini, E., deFrancesco, R., LaMonica, N. (1993) J. Virol. 67:40174026.

11. Wengler, G., Wengler, G. (1991) Virology 184:707-715.

12. Suzich, J. A., Tamura, J. K., Palmer-Hill, F., Warrener, P., Grakoui, A., Rice, C. M., Feinstone, S. M., Collett, M. S. (1993) J. Virol. 67:6152-6158.

13. Tamura, J. K., Warrener, P., Collett, M. S. (1993) Virology 193: 1-10.

14. Warrener, P., Tamura, J. K., Collett, M. S. (1993) J. Virol. 67:989-996.

15. Kim, D. W., Gwack, Y., Han, J. H., Choe, J. (1995) Biochem. Biophys. Res. Comm. 215: 160-166.

16. Jin, L., Peterson, D. L. (1995) Arch. Biochem. Biophys. 323:47-53.

17. Warrener, P., Collett, M. S. (1995) J. Virol. 69: 1720-1726.

18. Weiner, A. J., Brauer, M. J., Rosenblatt, J., et al., (1991) Virology 180:842-848.

19. Martell, M., Esteban, J. I., Quer, J., et al., (1992) J. Virol. 66:3225-3229.

20. Kato, N., Ootsuyama, Y., Sekiya, H., et al., (1994) J. Virol. 68:4776-4784.

21. Bukh. J., Miller, R. H., and Purcell, R. H. (1995) Seminars Liver Dis. 15:41-61.

22. Simmonds, P. (1995) Hepatology 21:570-583.

23. Okamoto, H., Kanai, N., Mishiro, S. (1992) Nucleic Acids Res. 20:6410.

24. Hayashi, N., Higashi, H., Kaminaka, K., et al., (1993) J. Hepatol. 17(suppl 3):S94-S107.

25. Silini, E., Bono, F., Cividini, A., Cerino, A., Bruno, S., Rossi, S., Delloni, G., Brugnetti, B., Civardi, E., Salvanesehi, L., and Mondelli, M. U. (1995): Hepatology 21:285-290.

26. Honda, M., Kaneko, S., Sakai, A., et al., (1994) Hepatology 20: 1144-1151.

27. Koizumi, K., Enomoto, N., Kurosaki, M., et al., (1995) Hepatology 22:30-35.

28. Kanazawa, Y., Hayashi, N., Miea, E., et al., (1994) Hepatology 20: 1121-1130.

29. Trepo, C., Habersetzer, F., Bailly, F., et al., (1994) Antiviral Res. 24: 155-163.

30. Shimizu, Y. K., Hijikata, M., Iwamoto, A., Alter, H. J., Purcell, R. H., Yoshikura, H. (1994) J. Virol. 68: 1494-1500.

31. Farci, P., Alter, H. J., Wong, D. C., Miller, R. H., Govindarajan, S., Engle, R., Shapiro, M., Purcell, R. H. (1994) Proc. Natl. Acad. Sci. USA 91:7792-7796.

32. Choo, Q-L., Kuo, G., Ralston, R., Weiner, A., Chien, D., Van Nest, C., Han, J., Berger, K., Thudium, K., Kuo, C., Knasopon, J., McFarland, J., Tabrizi, A., Ching, I., Moss, B., Cummins, L. B., Houghton, M., Muehmore, E. (1994) Proc. Natl. Acad. Sci. USA 91:1294-1298.

33. Duarte, E. A., Novella, I. S., Weaver, S. C., et al., (1994) Infect. Agents Dis. 3:201-214.

34. D'Souza, E. D. A., Grace, K., Sangar, C. V., Rowlands, D. J., and Clarke, B. E. (1995) J. Gen. Virol. 76: 1729-1736

35. Manabe, S., Fuke, I., Tanishita, O., Kaji, C., Gomi, Y., Yoshida, S., Mori, C., Takamizawa, A., Yoshida, I., Okayama, H. (1994) Virology 198:636-644.

36. Lin, C., Pragal, B. M., Groakoui, A., Xu, J., Rice, C. M. (1994) J. Virol. 68:8147-8157.

37. Han, D. S., Hahm, B., Rho, H-M., Jang, S. K. (1995) J. Gen. Virol. 76:985-993.

38. Gorbalenya, A. E., Donehenko, A. P., Koonin, V. and Blinov, V. M. (1988) Nature 333:22.

39. Gorbalenya, A. E., Koonin, E. V., Donehenko, A. P. and V. M. Blinov. 1989. Two related superfamilies of putative helicases involved in replication, recombination, repair and expression of DNA and RNA genomes. Nueleic Acid Res. 17:4713-4729.

40. Hodgman, T. C. 1988. A new superfamily of replicative proteins. Nature 333:22-23.

41. Linder, P., P. F. Lasko, P. Leroy, P. J. Nielsen, K. Nishi, J. Schnier, and P. P. Slonimski. 1989. Birth of the D-E-A-D box. Nature 337: 121-122.

42. Schmid, S. R., and P. Linder. (1992) D-E-A-D protein family of putative RNA helicases. Mol. Micro. 6:283-292.

43. Wassarman, D. A., and J. A. Steitz. (1991) Alive with DEAD proteins. Nature 349:463-464.

44. Lain, S., J. L., Riechmann, M. T. Martfn, and J. A. Garefa. 1989. Homologous potyvirus and flavivirus proteins belonging to a superfamily of helicase-like proteins. Gene 82:357-362.

45. Rikkonen, M., J. Peranen, and L. Kaariainen. 1994. ATPase and GTPase activities associated with Semliki Forest virus nonstructural protein nsP2. J. Virol. 68:5804-5810.

46. Mirzayan, C., and E. Wimmer. (1992) Biochemical studies on poliovirus polypeptide 2C: evidence for ATPase activity. Virology 199: 176-187.

47. Rodriguez, P. L., and L. Carraseo. (1993) Poliovirus protein 2C has ATPase and GTPase activities. J. Biol. Chem. 268:8105-8110.

48. Lain, S., M. T. Martfn, J. L. Riechmann, and J. A. Garefa. (1991) Novel catalytic activity associated with positive-strand RNA virus infection: Nucleic acid-stimulated ATPase activity of the plum pox potyvirus helicase-like protein. J. Virol. 65: 1-6.

49. Lain, S., J. L. Riechmann, and J. A. Garcia. (1991) RNA helicase: a novel activity associated with a protein encoded by a positive strand RNA virus. Nucleic Acids Res. 18:7003-7006.

50. Luckow, V. A., Summers, M. D. (1989) Virology 170:31-39.

51. Summers, M. D., and Smith, G. E. (1987) Texas Agricultural Experiment Station Bulletin, No. 1555.

52. Erikson, R. L., Collett, M. S., Erikson, E., Purchio, A. F. (1979) Proc. Natl. Acad. Sci. USA 76:6260-6265.

53. Houston, P., Kodadek, T. (1994) Proc. Natl. Acad. Sci. USA 91:5471-5474.

54. Raney, K. D., Sowers, L. C., Millar, D. P., Benkovic, S. J. (1994) Proc. Natl. Acad. Sci. USA 91 :6644-6648.

55. Bjornson, K. P., Amaratunga, M., Moore, K. J. M., Lohman, T. M. (1994) Biochemistry 33: 14306-14316.

56. Amersham Life Science, Product Specification Document 22.

What is claimed is:

1. A native, authentic, enzymatically active NTPase/RNA helicase protein produced by a process comprising the steps of:
a) expressing an NTPase/RNA helicase encoding nucleic acid of hepatitis C virus in a eukaryotic expression system such that a complete, authentic and native NTPase/RNA helicase protein is synthesized, said authentic and native NTPase/RNA helicase protein comprising amino acids 1027-1657;
b) extracting NTPase/RNA helicase protein from said eukaryotic expression system in an enzymatically active form of said protein; and
c) purifying said NTPase/RNA helicase protein such that the enzymatically active form of said protein is maintained.

2. The protein produced according to claim 1, said nucleic acid of hepatitis C virus in step a) corresponding to a human hepatitis C virus nucleic acid.

3. The protein produced according to claim 1, said nucleic acid of hepatitis C virus in step a) being derived from a genotype of the human hepatitis C virus nucleic acid.

4. The protein produced according to claim 1, wherein said nucleic acid of hepatitis C virus in step a) is a variant of the human hepatitis C virus.

5. The protein produced according to claim 1, said nucleic acid of hepatitis C virus in step a) encoding a complete NS3 coding region.

6. The protein produced according to claim 1, said nucleic acid of hepatitis C virus in step a) encoding a complete NS3 through NS5B coding region comprising amino acid residues from 1027 to 3011 of hepatitis C virus genome.

7. The protein produced according to claim 1, wherein said expression system is a recombinant baculovirus-insect cell expression system.

8. The protein produced according to claim 1, wherein the extracted protein is purified by immunoaffinity chromatography using antibodies specific for hepatitis C virus proteins.

9. The protein produced according to claim 1, having basal NTPase activity in the range of 0-200 min-¹ and RNA helicase activity greater than 0.001 min-¹.

10. The protein produced according to claim 1, having basa1 NTPase activity less than 150 min-¹ and RNA helicase activity greater than 0.005 min-¹.

11. A process for preparing native, authentic, enzymatically active NTPase/RNA helicase protein comprising the steps of:
a) expressing an NTPase/RNA helicase encoding nucleic acid of hepatitis virus in a eukaryotic expression system such that a complete, authentic and native NTPase/RNA helicase protein is synthesized, said authentic and native NTPase/RNA helicase protein comprising amino acids 1027-1657;
b) extracting NTPase/RNA helicase protein from said eukaryotic expression system in an enzymatically active form of said protein; and
c) purifying said NTPase/RNA helicase protein such that the enzymatically active form of said protein is maintained.

12. The process according to claim 11, said nucleic acid of hepatitis C virus in step a) corresponding to a complete NS3 coding region.

13. The process according to claim 11, said nucleic acid of hepatitis C virus in step a) corresponding to a complete NS3 through NS5B coding region.

14. A native, authentic, enzymatically active NTPase/RNA helicase protein product produced by a process comprising the steps of:
a) expressing a nucleic acid sequence in an expression system, thereby producing an enzymatically active, native, full length hepatitis C virus NTPase/RNA helicase protein that comprises the amino acid residues having sequence numbers from 1027 to and including 1657, wherein said expression system is a eukaryotic expression system;
b) extracting said protein from said expression system, such that the extracted protein is in an enzymatically active form;
c) purifying said extracted protein from step b) such that the purified protein is an enzymatically active, native, full-length hepatitis C virus NTPase/RNA helicase protein.

15. A method for assaying a compound for anti-viral activity against hepatitis C virus comprising:
a) providing enzymatically active, native, authentic hepatitis C virus NTPase/helicase protein;
b) contacting said protein with a compound suspected of inhibiting helicase activity; and
c) measuring inhibition of the helicase activity in said protein by said compound.

16. A method for assessing a compound for anti-viral activity against a flavivirus, comprising:
a) providing enzymatically active, native, authentic flavivirus helicase protein;
b) contacting said protein with a compound suspected of inhibiting helicase activity; and
c) measuring inhibition of the helicase activity in said protein by said compound.

17. A method as claimed in claim 15, wherein multiple compounds are assayed simultaneously.

18. A method for assaying a compound for anti-viral activity against hepatitis C virus comprising;
a) providing an enzymatically active, hepatitis C virus NTPase/RNA helicase protein;
b) providing a partially duplex substrate in which both strands are RNA and at least two nucleotides at the 3′ end of at least one RNA strand are not involved in base pairing and at least one of said RNA strands is detectably labeled;
c) exposing said NTPase/RNA helicase protein to said partially duplex RNA substrate in the presence of a putative antiviral compound;
d) capturing any detectably labeled single stranded release strand product of the interaction between said RNA helicase protein and said substrate with a capture system comprising a specific binding pair, one member of said specific binding pair being conjugated with an oligonucleotide having a nucleotide sequence complementary to said detectably labeled release strand and the other member of said specific binding pair being affixed to a solid support; and
e) quantitating detectable label present in said release strand, as a measure of the anti-viral activity of said compound.

19. A method according to claim 18, wherein the other member of said specific binding pair is affixed to a mobile solid support.

20. A method according to claim 18 in which said oligonucleotide of said capture system is DNA.

21. A method according to claim 20 in which said capture system comprises said oligonucleotide conjugated with biotin and agarose beads coated with streptavidin or a derivative thereof.

22. A method as claimed in claim 18, wherein multiple compounds are assayed simultaneously.
